(19) United States
(12) Patent Application Publication
(10) Pub. No.: US 2007/0032881 A1
(43) Pub. Date: Feb. 8, 2007

(54) Hernia prosthesis

(75) Inventor: James Browning, Glasgow (GB)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Mpathy Medical Devices Limited, Glasgow (GB)

(21) Appl. No.: 10/577,348

(22) PCT Filed: Nov. 1, 2004
(86) PCT No.: PCT/GB04/04620

§ 371 Date: Apr. 28, 2006

(30) Foreign Application Priority Data
Oct. 31, 2003 (GB) 0325442.2

(51) Int. Cl. A61F 2/02

(52) U.S. Cl. 623/23.72

(57) ABSTRACT

Prosthesis is provided for repairing or resisting the formation of bodily hernia in particular, for inguinal hernia repair or femoral hernia repair. The prosthesis comprises an inner surface and an outer surface, the inner surface forms at least one channel that is sized to receive an anatomical structure and allow it to pass through the prosthesis without substantial compression of the anatomical structure.

### FIELD OF THE INVENTION

The present invention relates to prostheses for repairing or resisting the formation of bodily hernia in particular, but not exclusively, for inguinal hernia repair or femoral hernia repair and a method of using said prostheses.

### DISCUSSION OF THE PRIOR ART

A hernia is due to an abnormal protrusion of an organ or part thereof through its containing structure, due to a rupture or weakening in a layer of fascia creating an aperture or a defect in the fascia which causes it to be less able to contain the organ or part thereof. Hernia can occur at various anatomical positions in the abdomen where there is a weakness in the muscle, and are classified according to the site in which they occur.

Two particular types of hernia are inguinal hernia and femoral hernia.

Inguinal hernia occur in the groin when a portion of bladder, bowel or membrane pushes through a weak spot in the abdominal musculature around or at the inguinal canal. The inguinal canal is an opening between layers of abdominal muscle near the groin through which the spermatic cord passes in the male. Typically, inguinal hernia is a male condition.

Two particular types of inguinal hernia occur, direct inguinal hernia and indirect inguinal hernia.

An indirect inguinal hernia passes through the internal ring of the inguinal canal, along the canal and, if the hernia is large enough, emerges through the external ring and in the male descends into the scrotum.

A direct inguinal hernia differs from an indirect inguinal hernia as it pushes its way directly forwards through the posterior wall of the inguinal canal. Occasionally, in unusual circumstances, a direct hernia becomes large enough to push its way through the external ring and then into the neck of the scrotum.

The femoral artery and vein enter the femoral triangle from beneath the inguinal ligament within a fascial tube termed the femoral sheath. The femoral canal is a small, almost vertically-placed gap in the medial part of the femoral sheath. The function of the femoral canal is to firstly act as a dead space for expansion of the distended femoral vein and secondly as a lymphatic pathway from the lower limb to the external iliac nodes.

The femoral canal is a potential point of weakness in the abdominal wall which may develop a femoral hernia. The canal is around 1 to 1.5 cm in length. As the female pelvis is of greater width than the male pelvis, the femoral canal can be somewhat larger in females and female femoral hernia are more common. A femoral hernia is a protrusion through the femoral canal. The hernia sac may extend through the femoral canal.

Hernia repair generally requires the contents of the hernia to be eased back into position and then for the weakened area to be repaired. Repair can be effected by tension or tension-free suturing of the tissue and muscle to strengthen the weakened area or occlude ruptured areas. Alternatively, the weakened or ruptured area can be reinforced using a portion of synthetic mesh.

Meshes for use in the treatment of an inguinal or femoral hernia typically consist of a flat portion of mesh for application over the hernia area. The mesh allows a tension free repair to be made of the weakened area. Such flat meshes have been provided with an aperture therein or may be cut by a surgeon to allow the mesh to be arranged around an anatomical structure which passes through the opening or defect in the tissue, muscle or organ wall requiring repair or support.

Alternatively, for a well circumscribed defect, e.g. a deep inguinal hernia or femoral hernia, the repair device may be an implantable prosthesis which stops the rupture hole of the hernia.

Implantable prostheses of the prior art include the Bard PERFIX plug™, Ethicon's Prolene Hernia System™, and Surgipro Hernia Mate plug and Patch™ or Atrium Self-forming plugs™.

The Bard PERFIX plug™ is one of the most popular plugs and comprises a surgical mesh fabric arranged to form around 8 leaves or petals, which are joined in a central region to create a multi-layered cone. The central portion of the plug is pushed into the defect and the leaves trimmed according to the size of the defect such that they stop the defect. As the leaves project from the central portion, these aid the retention of the plug in the defect. In addition, an overlay patch may be positioned over the plug which surrounds those tissues surrounding the inguinal canal. Surgipro Hernia Mate plug and Patch™ and Atrium Self-forming plugs™ also comprise several leaves and an overlay patch and work in a similar fashion to the Bard product.

Ethicon's Prolene Hernia System™ comprises a first overlay patch for placing around the inner ring of the inguinal canal, a central portion and a second overlay patch for placing around the outer ring of the inguinal canal. The central portion corresponds to both a portion of the first and second overlay patches such that it is held in the inguinal canal by the two patches to block the canal.

In use, the implantable prostheses of the prior art block the inguinal canal and prevent a hernia sac from protruding through the canal. The defects blocked most effectively by the prostheses are substantially circular in cross section, as multi-layer prostheses are inherently stiff and may not fully conform to variations in the defect. In some circumstances, when a prosthesis is in use, gaps may be potentially left between the prosthesis and the surrounding tissue, muscle or organ wall of the opening or defect.

This potential for gaps can be increased by anatomical structures which under normal circumstances pass through the inguinal canal, such as the spermatic cord, and protrude at the edge of the prosthesis and this causes difficulty in completely occluding the defect.

To improve the flexibility of conventional prostheses and thus minimise the potential gaps between the prostheses and surrounding tissues, some prostheses include pleats moulded into the body of the prostheses. Although, such pleats may to some extent accommodate anatomical structures which pass through the defect in the tissue, as such a prosthesis relies on a push fit of the prosthesis into the defect and radial expansion of the leaves of the prosthesis against the tissues surrounding the defect to hold the implant in place, such a prosthesis will compress anatomical structures between the prosthesis and the surrounding tissue. This compression can result in a significant pressure being experienced by an anatomical structure.

Significant pressure is a pressure which causes distortion, compression or full or partial collapse of an anatomical structure. For example, in particular examples where a conventional prosthesis is used to treat inguinal hernia, the spermatic cord is squeezed between the prosthesis and the tissues surrounding the aperture and this squeezing may cause pain or even damage to the spermatic cord. This can lead to discomfort for the patient and might lead to long term damage to the structure(s) being compressed and may cause ischaemia of a distal organ. For example, where the anatomical structure includes the spermatic cord, ischaemia of the testes may occur as a result of compression of the artery and/or vein along with the spermatic cord.

According to the present invention there is provided a prosthesis for repair or to resist the formation of hernia of the abdominal wall, the prosthesis comprising at least an outer surface and an inner surface wherein, the inner surface forms at least one channel through which, in use, an anatomical structure may pass when the prosthesis is in place in the body without substantial compression of said anatomical structure.

The channel may be an indentation in the outer surface of the prosthesis.

Preferably the channel is formed along the outer surface of the prosthesis.

An advantage of a prosthesis of the present invention is that by providing such a channel in the prosthesis, pressure on an anatomical structure passing through the channel can be minimised. A reduction or complete removal of the pressure on an anatomical structure should minimise damage and/or discomfort caused by compression of anatomical structures passing through the defect being repaired and minimise the rupture or protrusion of a hernia sac through the defect.

In a preferred embodiment of the prosthesis the inner surface defines a scalloped channel.

A scalloped channel is formed by the intersection or indentation of a cylinder with the outer surface of the prosthesis.

In a particularly preferred embodiment of the prosthesis the channel has a substantially semi-circular edge in cross section, such that the inner surface is substantially curved as it interfaces with the anatomical structure which the channel receives.

Preferably, in use, the prosthesis is always wholly contained within the extra peritoneal compartment of the abdominal wall.

Preferably the prosthesis is suitable for use in the treatment of abdominal hernia. More preferably the prosthesis is suitable for treatment of inguinal or femoral hernia.

In an embodiment of the prosthesis, the prosthesis is provided for repairing or resisting the formation of an inguinal hernia, the channel being sized to accommodate a spermatic cord without substantial compression of the spermatic cord.

In a preferred embodiment of the prosthesis, wherein the prosthesis is for use in repairing or resisting the formation of an inguinal hernia, the prosthesis has a longitudinal length or depth in the range 1 cm to 5 cm. More preferably the prosthesis has a longitudinal length in the range of between 2 cm to 3 cm.

In a preferred embodiment of the prosthesis for use in repairing or resisting the formation of an inguinal hernia, the prosthesis is of width or diameter in the range 0.5 cm to 7 cm. In a particular embodiment the prosthesis is of width or diameter in the range 1 cm to 4 cm.

In a particularly preferred embodiment of the prosthesis for repairing or resisting the formation of an inguinal hernia, the prosthesis has a truncated conical shape wherein the outer surface of the prosthesis is formed by the conic surface.

A prosthesis of truncated conical shape in which a first end of the prosthesis has a diameter less than that of a second end has the advantage that the prosthesis can be pushed first end into the defect, to plug the defect more easily.

In a particularly preferred embodiment, the prosthesis is of truncated conical shape and further comprises a semi-circular channel extending from a first end of the prosthesis to a second end of the prosthesis, the first end having a diameter less than the second end, wherein the semi-circular channel is present in the conic outer surface of the prosthesis such that in cross-section a crescentic shaped prosthesis is provided.

In an embodiment wherein the prosthesis has a truncated conical shape, the diameter of the widest end of the prosthesis, the second end, is preferably in the range 1 cm to 7 cm and the diameter of the narrowest end, the first end, is preferably in the range 0.5 cm to 4 cm.

The channel receiving the anatomical structure can have any suitable cross sectional shape such as a semi-circular cross section. In an embodiment of the prosthesis for repairing or resisting the formation of an inguinal hernia, the channel is sized in the range 0.5 cm to 3 cm in width and depth or where the channel of such an embodiment of the prosthesis is of circular or substantially circular cross section, for example semi-circular cross section, the channel is in the range 0.5 cm to 3 cm in diameter.

In another embodiment of the prosthesis, the prosthesis is provided for repairing or resisting the formation of a femoral hernia. In such an embodiment the length of the prosthesis is in the range 1 cm to 5 cm, the width of the prosthesis is in the range 0.5 cm to 7 cm and the channel is sized to receive at least one of a femoral vein or other anatomical structure.

In a preferred embodiment of a prosthesis provided for repairing or resisting the formation of a femoral hernia the prosthesis is of truncated conical shape.

In an alternative embodiment the prosthesis provided for repairing or resisting the formation of femoral hernia is of triangular prism shape. In another embodiment, in cross section, the prosthesis is substantially arrowhead shaped having two outer accurate sides which extend from a base towards each other to form a point. Preferably the point is rounded. Alternatively, the prosthesis is substantially D shaped with the accurate sides forming a more rounded arched point.

In a particular embodiment the prosthesis is formed from a number of component prosthetic parts which together form the complete prosthesis of the first aspect of the invention.

In an embodiment of the prosthesis formed from at least two component parts, the parts may include means to attach the parts to each other to form the complete prosthesis.

It can be envisaged that the component prosthetic parts are of suitable shape such that in combination they provide a prosthesis which provides a channel able to receive an anatomical structure.

Typically the prosthesis is formed from resilient material such that the prosthesis can be flexed to open the access to the channel.

Suitably the prosthesis may be constructed of synthetic polymer which may be absorbable or non-absorbable, mesh material formed from synthetic polymer, solid material, foam or hydrogel. Suitable synthetic polymers include, but are not limited to, polyester, polypropylene, PTFE, Mersilene, MPathy-Mesh™ and Mini-Mesh™ (available from MPathy Medical Devices Limited, UK).

The prosthesis may be formed from rolls of mesh and/or comprises cross members to provide the prosthesis with strength to resist compression. The prosthesis may be formed from plastics material. In a particular embodiment the foam used to construct the prosthesis is polyurethane.

This is advantageous in that the channel may be formed such that, in use, the prosthesis may be flexed from its rest position to an open position to increase the width of the access to the channel enabling an anatomical structure to be more easily received by the channel. The prosthesis may then be released to return to its rest position wherein the anatomical structure is substantially enclosed by the channel when the prosthesis is located in the defect.

An anatomical structure may be partially received and enclosed by the channel of the prosthesis. Typically an anatomical structure may be partially received and enclosed by the channel such that at least 30% of the circumference of the anatomical structure is surrounded by the prosthesis.

The channel of the prosthesis is sized such that in use an anatomical structure may pass, when the prosthesis is in place in the body, without substantial compression of said anatomical structure by the prosthesis. Substantial compression of the anatomical structure is compression which causes pain to the patient or ischaemia of a distal organ. Preferably the width of the anatomical structure, which in use passes through the channel, is compressed less than 70%, even more preferably less than 50%, yet more preferably less than 40%, even more preferably less than 30%, even more preferably less than 20%, yet more preferably less than 10%, even more preferably less than 5%, even more preferably less than 3%, most preferably less than 1% by the channel of the prosthesis.

The level of compression experienced by the anatomical structure by the prosthesis when the anatomical structure passes through the channel of the prosthesis is preferably not more than venous pressure. Venous pressure is typically in the range 2 to 10 mm Hg.

In one embodiment of the prosthesis a single channel, sized to receive at least one anatomical structure, is provided. In another embodiment two channels each sized to receive at least one anatomical structure, are provided. Each channel may be differently sized to receive at least one anatomical structure in order to maximise the support provided by the prosthesis while allowing the structure(s) to pass through the one or more defined channels in the prosthesis.

A plurality of channels, each channel sized to receive one or more anatomical structures, may be received by the prosthesis.

In a preferred embodiment of the present invention the prosthesis further comprises at least one flange provided on either one or both ends of the prosthesis. The provision of a flange on the prosthesis is advantageous as it aids location of the prosthesis in the body and may provide additional support to tissue, muscle or an organ wall surrounding the defect. In particular embodiments, the flange extends from the prosthesis such that, in use, the flange provides an inferomedial extension to the prosthesis. For example, if a prosthesis of the invention further comprising a flange is used to plug an inguinal canal, a first end of the prosthesis is positioned at the internal inguinal ring of the inguinal canal and a second end of the prosthesis is positioned at the external ring of the inguinal canal and a flange present on the second end of the prosthesis, can inferomedially extend from the prosthesis around the external ring.

The flange may be provided by a layer of synthetic mesh. Alternatively, the flange may be formed from a plurality of layers of synthetic mesh.

The layer(s) of mesh may overlap each other. Moreover, the layer(s) of mesh may be of any desired shape to support the surrounding tissue, muscle or organ wall.

It is advantageous for the flange to be constructed of mesh. The mesh has minimal mass density in relation to its volume. In a preferred embodiment the flange is constructed of Mini-Mesh™.

A flange portion may contain structures or regions capable of receiving sutures or other fixing means to secure the flange around the anatomical structures received by the channel and/or to secure the flange to the surrounding tissue. The flange may comprise more than one portion of material. For example, a flange may comprise two or more portions which can be arranged around an anatomical structure. The two portions may attach to each other or overlap each other to form an extended region of support to a hernia. The portions of the flange which overlap each other may be formed of thinner material such that the overlapped region has the same thickness as the non-overlapped region of the flange.

In a particular embodiment of the prosthesis, the prosthesis has a crenated outer surface. The crenated outer surface allows the prosthesis to grip the tissues surrounding the prosthesis and aids retention of the prosthesis, in position, in the body.

In a second aspect of the present invention there is provided a kit of parts including a prosthesis according to the first aspect of the invention and synthetic mesh for overlaying the prosthesis when the prosthesis is positioned in the body. The kit may also include instructions as to the way in which the components of the kit are to be used.

According to a third aspect of the invention there is provided a method for treating a hernia comprising the steps:
exposing the hernia defect
providing a prosthesis wholly in the extra peritoneal compartment of the abdominal wall to fill the defect but providing a relatively pressure free passage of an anatomical structure past the prosthesis.

The method is for treatment of abdominal hernia. Typically the method may be used for treatment of inguinal or femoral hernia.

The method may further include the step of fixing the prosthesis to the margins of the defect. One example of the way in which the prosthesis may be fixed to the margins of the defect is by suturing.

The method may further include the step of overlaying the prosthesis with mesh.

The method preferably uses the prosthesis of the first aspect of the invention or the kit of the second aspect of the invention.

Preferred features of each aspect of the invention are as for each of the other aspects mutatis mutandis unless the context demands otherwise.

### BRIEF DESCRIPTION OF THE DRAWINGS

Embodiments of the present invention will now be discussed, by way of example only, with reference to the accompanying figures in which;

FIG. 1 shows a perspective view of an embodiment of a prosthesis of the invention from a second end;

FIG. 2 shows a perspective view of an embodiment of a prosthesis of the invention from a first end;

FIG. 3 shows a perspective view of an embodiment of a prosthesis of the invention in use;

FIG. 4 shows an embodiment of a prosthesis which further includes a flange provided at one end of the prosthesis;

FIG. 5 shows an indirect inguinal hernia;

FIG. 6 shows a hernia repaired using a conventional prosthesis of the prior art;

FIG. 7 shows an illustration of the anatomy around the inguinal canal; and

FIG. 8 shows an illustration of the anatomy around the femoral canal.

### DESCRIPTION OF ILLUSTRATIVE EMBODIMENTS

The invention is directed to an implantable prosthesis for repairing or resisting the formation of bodily hernia, in particular to plug or stop any aperture in the body in which a structure is required to pass through or adjacent to the aperture. For example, the prosthesis may be used to plug the inguinal canal or the femoral canal. In these embodiments, provided by way of example only, the prosthesis has a channel through which an anatomical structure, such as a spermatic cord or femoral vein, may pass without substantial compression of the anatomic structure.

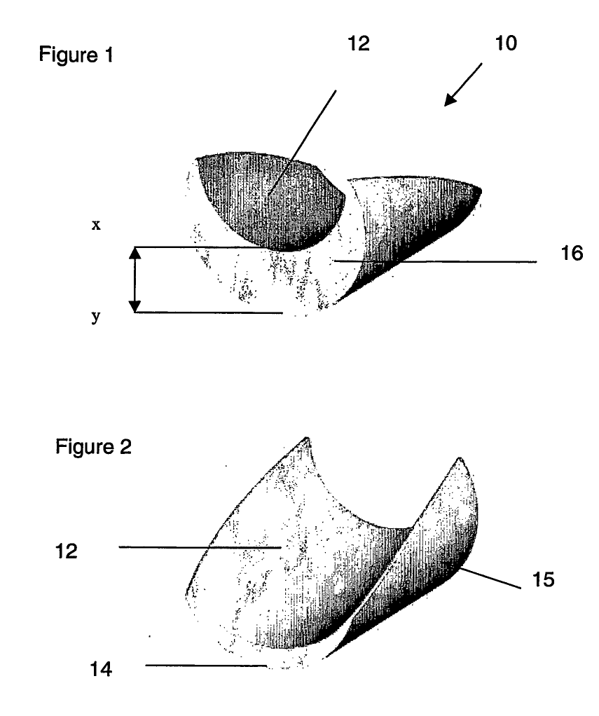

As shown in FIGS. 1 and 2, in one embodiment, the prosthesis 10 is a truncated cone having a first end 14 and a second end 16, wherein the diameter of the first end is less than the diameter of the second end 16, and an outer conic surface 15 extends between the ends. An inner surface 12 forming a channel is defined by a substantially scalloped portion removed from the outer surface of the truncated conical prosthesis. It can be envisaged that the scalloped portion is formed by the removal of a cylindrical portion which intersects the outer conical surface 15 to create a prosthesis of crescential cross-section. The prosthesis resembles a wedge shape being narrower at the first end and widest at its second end. An anatomical structure may pass through the prosthesis whilst being partially surrounded by the prosthesis to minimise the pressure or compression exerted on the anatomical structure.

It will be understood that the cross section of the channel may be formed by at least one straight edge such that the inner surface has a straight portion in cross section, for example a box section channel or at least one curved edge, to form a semi-circular channel or other shapes as should be apparent to one skilled in the art.

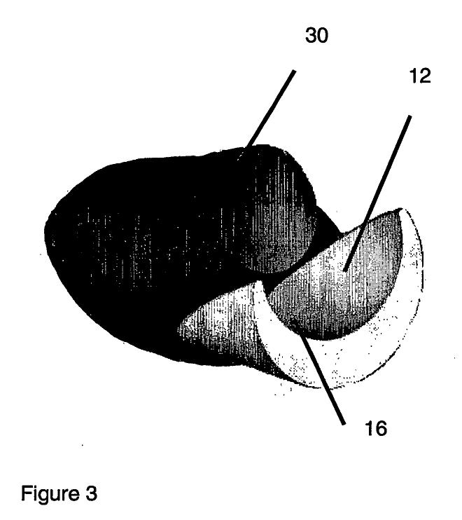

The channel 12 in the outer conical surface 15 of the prosthesis 10 is sized to receive an anatomical structure(s) which passes through the defect to be repaired or supported. As shown in the illustrated embodiment, FIG. 3, the channel formed by inner surface 12 receives an anatomical structure 30 such that the anatomical structure is partially located in the channel. The channel minimising the compression of the anatomical structure against the edges of the defect when, in use, the prosthesis is located in the body.

In the embodiment of the prosthesis illustrated in FIGS. 1 to 3 for use in repair of an inguinal hernia the prosthesis is of truncated conical shape with a semi-circular channel removed from the conical surface such that the prosthesis is substantially a wedge shape extending from a first end 14 of minimal depth to a second end of diameter of around 19 mm. The channel is of around 15 mm in depth at the second end, such that in cross-section the second end is crescential in shape with a maximum depth (x-y see FIG. 1) of 7 mm. The length of the prosthesis between the first and second ends is around 23 mm.

The portion removed from the truncated conical prosthesis to provide a channel can be in the range of 5 mm to 20 mm in width and depth. Although in the embodiment shown in FIGS. 1 to 3, the channel is substantially semi-circular in cross section, the channel may be of any shape. In addition, more than one channel may be present in the prosthesis, each channel being able to receive a particular anatomical structure.

Typically the prosthesis is in the range 1 cm to 5 cm in length between the ends and around 1 cm to 4 cm in width and depth.

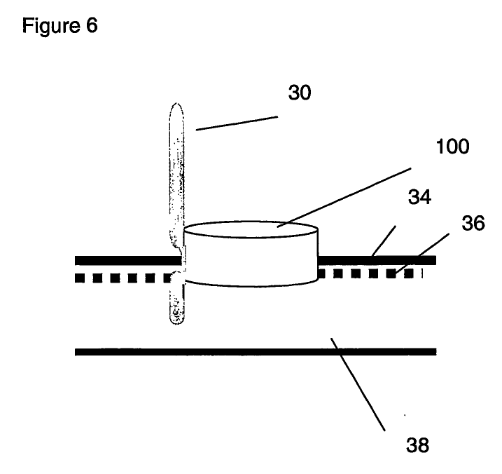

As shown in FIG. 3, in use, an anatomical structure 30 is received by the channel 12, the channel indenting the conical surface of the prosthesis and linking the first and second ends 14 and 16, such that the anatomical structure can pass from one end of the prosthesis to the other without substantial compression. This differs from the conventional prosthesis 100, illustrated in FIG. 6, which lacks a channel. As illustrated in FIG. 6 when a conventional prosthesis is in use to plug a defect, for example in abdominal wall muscle 36 and fat 34 through which a hernia 32 of viscos 38 protrudes, an anatomical structure 30, such as a spermatic cord, is located between the prosthesis 100 and the edge of the defect. As the prosthesis 100 lacks a channel and the prosthesis is pushed into the defect, the anatomical structure is compressed.

The prosthesis and further the flange portion may be formed from a range of material including, but not limited to, polyester, polypropylene, PTFE, Mersilene, MPathy-Mesh™ or Mini-Mesh™ (available from MPathy Medical Devices Limited, UK).

The prosthesis may be formed using suitable construction techniques, for example knitting and/or weaving of monofilament or multifilament yarns, moulding, ultrasonic, induction, vibration, infrared or laser welding.

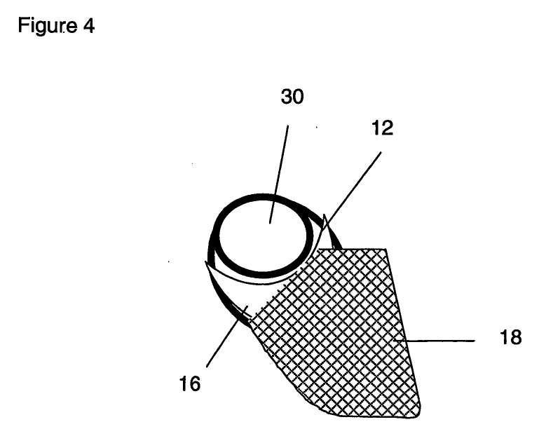

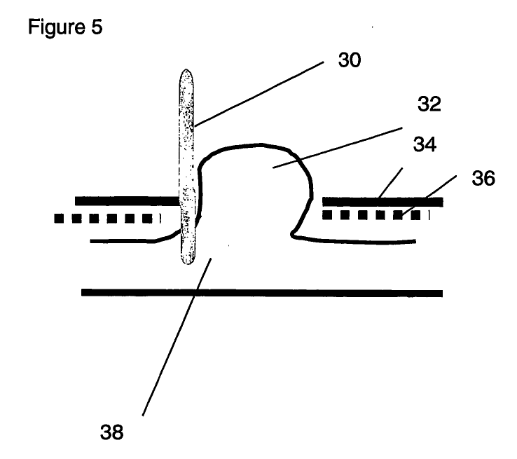

As illustrated in FIG. 4, the prosthesis of the present invention may further comprise a flange 18. The flange may extend laterally from at least a first or second end or both ends of the prosthesis.

As shown in FIG. 4, when the prosthesis is located in the defect, the flange 18, which extends from the second end of the prosthesis, can aid the positioning of the prosthesis, in the inguinal canal. Further, the flange may be formed from mesh and extend from the prosthesis such that when the prosthesis is implanted in the body the mesh extends to the musculature surrounding the inguinal canal and provides support thereto. In particular embodiments, the flange can extend from the prosthesis inferomedialy, which aids the use of the prosthesis in the treatment of direct inguinal hernia.

The flange may include more than one layer of mesh and said layers may overlap each other. Moreover, the flange may include cut out potions to allow it to be placed around or over protruding structures or attachment means to attach the flange to itself and/or tissue, muscle etc. Such attachment means include sutures or other fixing means.

In embodiments wherein a flange is provided on both ends of the prosthesis, the flange, when the prosthesis is in use, may be provided around the internal ring and external ring of the inguinal canal such that the tissue and fascia around the inguinal ring is sandwiched between at least two layers of mesh. The flange thus supports the tissue and/or fascia and minimises the likelihood of organs or structures rupturing or protruding through the tissue and/or fascia.

An embodiment of the prosthesis of the first aspect of the invention can be utilised to repair or resist the formation of an inguinal canal.

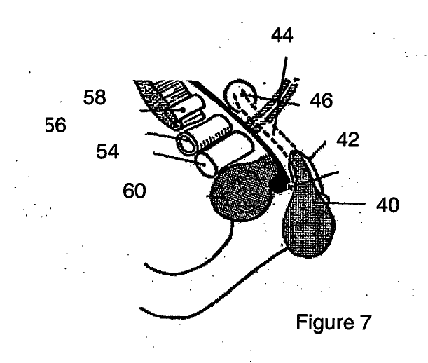

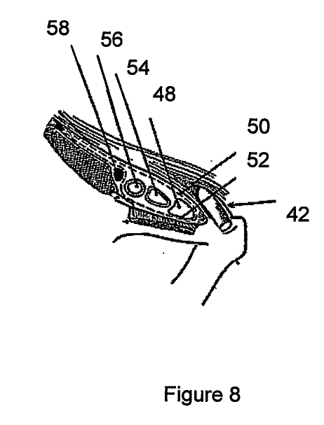

As illustrated in FIGS. 7 and 8 the sac of an indirect inguinal hernia 40 may extend from the external ring 42 of the inguinal canal 44. The inguinal canal extending between the external ring 42 and an internal ring 46.

In use, a prosthesis is inserted into the inguinal canal such that a first end of the prosthesis is positioned at the internal inguinal ring 46 and the second end is positioned at the external ring 42 of the inguinal canal. When located in the inguinal canal 44 the prosthesis acts to minimise the protrusion of organs or the other body parts through the inguinal canal, but as the prosthesis includes a channel, there is provided a passage for selected anatomical structures, such as the spermatic cord, to pass through the prosthesis without being substantially compressed by the prosthesis or between the prosthesis and the surrounding tissue.

To aid the fixation of the prosthesis in the inguinal canal the prosthesis may be crenated on its outer surface. Such crenations will project from the outer surface of the prosthesis into the surrounding tissue and minimise the movement of the prosthesis once it has been suitably positioned.

An embodiment of the prosthesis of the invention may be used to repair or resist the formation of a femoral hernia. As illustrated in FIGS. 7 and 8 the femoral canal 48 lies between the fascia transversalis 50 and fascia iliaca 52 with the femoral vein 54, femoral artery 56 and femoral nerve 58 being present to one side of the femoral canal. As shown in FIG. 7, a sac of a femoral hernia 60 may extend along and pass out of the femoral canal.

In use, an embodiment of the prosthesis for treatment of femoral hernia may be inserted into the femoral canal to minimise the protrusion of the hernia sac through the femoral canal. During insertion of the prosthesis into the femoral canal, the channel of the prosthesis is orientated such that expansion of the femoral vein is into the channel of the prosthesis. Thus, in contrast to conventional prosthesis, the compression of the expanded vein against the prosthesis and/or the surrounding tissue will be minimised. In addition, the channel will still provide for movement in the lymphatic system from a lower limb to external iliac nodes.

In one embodiment, a prosthesis of the present invention, for use in plugging the femoral canal, is substantially of triangular prism shape in cross section such that it is shaped to fit into the femoral canal. In another embodiment, in cross section, the prosthesis is substantially arrowhead shaped having two outer accurate sides which extend from a base towards each other to form a point. Preferably the point is rounded. Alternatively, the prosthesis is substantially D shaped with the accurate sides forming a more rounded arched point. In each embodiment a channel is provided in the outer surface of the prosthesis to receive the femoral vein when it is expanded. When the prosthesis is substantially arrowhead or D shaped, it is preferred that the base portion is indented towards the point to receive an anatomical structure.

The prosthesis is sized such that it can be suitably located into the femoral canal. In particular embodiments the prosthesis is sized such that it is of length in the range 1 cm to 5 cm, of width at a first end for insertion into the femoral canal in the range 0.5 cm to 3 cm and a second end at 0.5 cm to 5 cm.

The channel need only be an indentation in the outer surface of the prosthesis to receive the femoral vein when expanded such that the pressure exerted on the vein, during expansion of the vein, by the prosthesis is minimised.

As discussed above, a prosthesis for use in treating femoral hernia may further include a flange at either or both ends of the prosthesis, wherein the flange extends around the femoral canal and thus supports the surrounding tissue or fascia. As previously discussed such a flange may also contain cutouts to accommodate structures such as the femoral nerve and/or artery.

The prosthesis of the present application has been designed to take into consideration the anatomical structures and properties of the inguinal and femoral canal to minimise the disruption of these structures following location of the prosthesis.

Various modifications can be made without departing from the scope of the invention, for example, flanges extending from the faces of the prosthesis, as discussed above, may be formed from material with memory, such that following placement in the body the flanges move from a collapsed position to an extended position to secure the prosthesis in the body.

1. A prosthesis for repair or to resist the formation of an abdominal wall hernia comprising at least an outer surface and an inner surface wherein the inner surface forms at least one channel which, in use, partially receives and encloses an anatomical structure such that at least 30% of the circumference of the anatomical structure is surrounded by the prosthesis and the an anatomical structure may pass through the prosthesis when the prosthesis is in place in the body without substantial compression of said anatomical structure.

2. The prosthesis as claimed in claim 1 of width in the range 0.5 cm to 7 cm.

3. The prosthesis as claimed in claim 1 of longitudinal length in the range 1 to 5 cm.

4. The prosthesis as claimed in claim 1 wherein the channel is of dimensions in the range 0.5 cm to 3 cm in width and depth.

5. The prosthesis as claimed in claim 1 wherein the inner surface defines a scalloped channel.

6. The prosthesis as claimed in claim 1 wherein the channel of the prosthesis is sized such that in use an anatomical structure may pass, when the prosthesis is in place in the body, the width of the anatomical structure being compressed less than 70%.

7. The prosthesis as claimed in claim 1 wherein the prosthesis has a truncated conical shape.

8. The prosthesis as claimed in claim 7 wherein the diameter of the truncated conical shaped prosthesis at the widest end of the prosthesis is in the range 0.5 cm to 7 cm and the diameter of the narrowest end is in the range 0.5 cm to 4 cm.

9. The prosthesis as claimed in claim 1 wherein the prosthesis further comprises at least one flange which extends from the prosthesis.

10. The prosthesis as claimed in claim 9 wherein the flange is a portion of synthetic mesh.

11. Use of a prosthesis as claimed in claim 1 to repair or to resist the formation of an abdominal hernia.

12. Use of a prosthesis as claimed in claim 1 to repair or to resist the formation of an inguinal hernia, the channel being sized to receive a spermatic cord without substantial compression of the spermatic cord.

13. Use of a prosthesis as claimed in claim 1 to repair or to resist the formation of femoral hernia the channel being sized to receive a femoral vein.

14. A kit including a prosthesis according to claim 1 and synthetic mesh to overlay the prosthesis.

15. A method for treating hernia comprising the steps:
exposing the hernia defect
providing a prosthesis wholly in the extra peritoneal compartment of the abdominal wall to fill the defect but which provides a relatively pressure free passage of an anatomical structure past the prosthesis.

16. The method of claim 15 wherein the method is for treatment of inguinal or femoral hernia.

17. The method of claim 15 wherein the prosthesis provided to fill the defect is the prosthesis of claim 1.
